(19) United States
(12) Patent Application Publication
(10) Pub. No.: US 2024/0309438 A1
(43) Pub. Date: Sep. 19, 2024

(54) RARE NUCLEIC ACID DETECTION

(71) Applicant: Harbinger Health, Inc., Cambridge, MA (US)

(72) Inventors: Anthony P. Shuber, Northbridge, MA (US); Jennifer L. Steele, Burlington, MA (US)

(21) Appl. No.: 18/514,333

(22) Filed: Nov. 20, 2023

Related U.S. Application Data
(63) Continuation of application No. 18/128,698, filed on Mar. 30, 2023.
Application No. 18/128,698 is a continuation of application No. 16/007,541, filed on Jun. 13, 2018, now abandoned.
(60) Provisional application No. 62/634,250, filed on Feb. 23, 2018.
(60) Provisional application No. 62/526,091, filed on Jun. 28, 2017.
(60) Provisional application No. 62/519,051, filed on Jun. 13, 2017.

(51) Int. Cl. C12Q 1/6848; C12N 9/22; C12N 15/11; C12Q 1/6827; C12Q 1/6886

(52) U.S. Cl. CPC C12Q 1/6848; C12N 9/22; C12N 15/11; C12Q 1/6827; C12Q 1/6886; C12N 2310/20; C12N 2800/80; C12Q 2600/106

(57) ABSTRACT

Methods for detecting rare mutations in DNA include obtaining a sample comprising a target nucleic acid, binding a protein to the target nucleic acid in a sequence-specific manner, digesting non-target nucleic acid in the sample, and detecting the target nucleic acid. The method may include amplifying the target nucleic acid with at least one primer with, e.g., a phosphorothioate bond that is resistant to degradation by a nuclease to yield an amplicon that includes a copy of the target nucleic acid and a terminal portion that is resistant to degradation by the nuclease. Preferably digesting the non-target nucleic acid includes exposing amplicons to the nuclease. The nuclease digests the non-target nucleic acid while the amplicon that includes the copy of the target nucleic acid is protected by the terminal portions and the bound protein.

### CROSS-REFERENCE TO RELATED APPLICATIONS

This application claims the benefit of, and priority to, U.S. Provisional Application 62/634,250, filed Feb. 23, 2018, U.S. Provisional Application 62/526,091, filed Jun. 28, 2017, and U.S. Provisional Application 62/519,051, filed Jun. 13, 2017, the contents of each of which are incorporated by reference.

### TECHNICAL FIELD

The disclosure relates to molecular genetics.

### BACKGROUND

Laboratories are increasingly using DNA and RNA for clinical analysis. For example, DNA can reveal whether a person has a disease-associated mutation, or is a carrier of a hereditable disease. Additionally, fetal DNA can be studied to detect inherited genetic disorders and aneuploidy. However, a consistent challenge in accessing actionable genomic information lies in existing approaches to detecting very rare mutations, i.e., mutant alleles of DNA present only in very small frequencies among large populations of DNA.

Various clinical assays have been proposed for detecting mutations including, for example, tests based on hybridization of fluorescent probes and tests based on DNA sequencing. Typical DNA sequencing assays include the use of next-generation sequencing (NGS) platforms to capture, amplify, and sequence a subject's DNA. However, typical NGS platforms face a number of challenges. Detecting rare mutations in samples that also contain an abundance of wild-type DNA requires successfully amplifying rare DNA species. Given the stochastic nature of PCR, the ability to amplify rare fragments has been a challenge. Other detection methods, such as using fluorescent probe hybridization, face similar challenges. For example, when a mutant is present in quantities as low as hundredths of a percent of copies present, probe assays may miss the mutant entirely.

### SUMMARY

The invention provides methods for detecting DNA sequence that is present in low abundance in a sample. Methods of the invention utilize a protein that binds, in a sequence-specific manner, to the rare species (e.g., a nucleic acid containing a mutation) to protect the rare species, while unprotected, off-target nucleic acid is digested. Methods of the invention have particular applicability in liquid biopsy, the detection of mutations in plasma. The use of binding proteins takes advantage of kinetics more similar to enzyme kinetics than to DNA hybridization, thus allowing a significantly-higher capture rate. In particular, RNA-guided binding proteins, such as a Cas endonuclease, can bind to, and protect, mutation-containing nucleic acid even when the mutation is only present as a small fraction of the sample. Thus, methods of the invention are useful when analyzing tumor/cancer mutations as may be found among circulating, cell-free DNA in a blood or plasma sample.

In a preferred method, CRISPR/Cas systems using guide RNAs specific for a mutation is introduced to the sample under conditions such that nucleic acid containing the mutation is protected from exonuclease digestion while non-target nucleic acid is digested by an exonuclease. When used according to methods of the invention, Cas endonuclease—whether catalytically active or inactive—will bind to a target consistent via a guide RNA and will protect that target (i.e., stay bound) for at least long enough that a promiscuous exonuclease can be reliably used to digest unbound, non-target nucleic acid. By protection of the target with digestion of the non-target, a sample is effectively enriched for the target, and those remaining target fragments are captured, stored, isolated, preserved, detected, sequenced, or otherwise assayed with success that would be unobtainable without methods of the invention.

Preferred embodiments of the invention make use of an amplification step that uses at least on primer that is resistant to exonuclease activity. The mutation of interest may lie in a region flanked by PCR primer binding sites. Methods may include using a corresponding pair of PCR primers in which copies of one of the primer pair includes a phosphorothioate backbone linkage. The target region is amplified using the primer to yield a plurality of amplicons in which one strand of the amplicon includes one or more phosphorothioate linkages near one end of the strand. Among the amplicons, the mutation of interest will be present in an allele fraction approximating an allele frequency of the mutation in the original sample. An RNA-guided binding protein such as a Cas complex (e.g., a Cas endonuclease or a catalytically inactivated Cas endonuclease complexed with a guide RNA) is introduced with a guide RNA that binds to the mutation of interest. The Cas complex binds to amplicons that include the mutation. An exonuclease is introduced that digests DNA. However, those amplicons that include the mutation and are bound by the Cas complex will not be digested by the exonuclease. For those amplicons, one end of the fragment will be protected from the exonuclease by the phosphorothioate linkages, while the other end will be protected by the bound Cas complex. After digestion with the exonuclease, the only fragments that remain will be fragments that contain the mutation of interest. Those fragments can then be detected by a suitable assay, such as sequencing, gel electrophoresis, a probe-based assay, or a subsequent amplification such as a rolling circle amplification (e.g., followed by probing with, e.g., molecular beacons).

Methods and related kits described herein are useful to detect the presence of mutation in a sample. Due to the nature by which a protein such as a Cas complex binds to a target, methods may be used even where the target is present only in very small quantities, e.g., even as low as 0.01% frequency of mutant fragments among normal fragments in a sample (i.e., where a plurality of homologous fragments includes about 500.000 wild-type fragments and about 50 mutant fragments). Thus, methods of the invention may have particular applicability in discovering very rare yet clinically important information, such as mutations that are specific to a tumor and even may be used to detect specific mutations among cell-free DNA, such as tumor mutations among circulating tumor DNA.

In certain aspects, the invention provides methods of detecting a target nucleic acid. Preferred methods include obtaining a sample comprising a target nucleic acid, binding a protein to the target nucleic acid in a sequence-specific manner, digesting non-target nucleic acid in the sample, and detecting the target nucleic acid. Methods may include amplifying the target nucleic acid with at least one primer that is resistant to degradation by a nuclease to yield an amplicon that includes a copy of the target nucleic acid and a terminal portion that is resistant to degradation by the nuclease. Preferably, digesting the non-target nucleic acid includes exposing amplicons to the nuclease. The nuclease digests the non-target nucleic acid while the amplicon that includes the copy of the target nucleic acid is protected by the terminal portions and the bound protein. In certain embodiments, the primer that is resistant to degradation by the nuclease includes a phosphorothioate linkage.

In preferred embodiments, the sample is a liquid biopsy sample. The target nucleic acid may include a mutation specific to a tumor. The tumor mutation may be present at no more than about 0.01% among matched normal, non-tumor nucleic acid.

In preferred embodiments, the protein is an RNA-guided protein complexed with a guide RNA, in which the guide RNA has a targeting portion that hybridizes to a complementary portion in the copy of the target nucleic acid. The RNA-guided protein may be a Cas endonuclease or a catalytically deficient homolog thereof.

The target nucleic acid may include a mutation, and the sample may also include homologous non-mutated (e.g., wild-type) nucleic acid, and the digesting step may include digesting the homologous non-mutated nucleic acid, amplified copies thereof, or both.

The result of the digesting may be a product that includes one or more fragments that include copies of the rare mutation. That reaction product may be an input to an assay that facilitates detection or analysis of the mutation. For example, in some embodiments, the fragments containing the rare mutations are used as input into an amplification, such as a rolling circle amplification. In such embodiments, the isolated fragment(s) containing the rare mutation may be incorporated into a circularized template that may, in-turn, be amplified by a rolling circle amplification. The rolling circle amplification may be beneficial because it can proceed using either of those same primers used in an earlier amplification step, it can produce a large number of copies of the rare mutation, or both.

In certain embodiments, the sample is from a patient, and the method may include providing a report describing the mutation as present in the patient. The method may further include identifying a treatment based on the presence of the mutation in the patient and including the identified treatment option in the report.

In most preferred embodiments, the digesting is performed with an exonuclease, and the protein is a Cas endonuclease complexed with a guide RNA, in which the guide RNA comprises a targeting portion that hybridizes to a complementary portion in the target nucleic acid. In some embodiments, the protein comprises a transcription-activator like effector (TALE).

In an exemplary embodiment, the method includes: amplifying the target nucleic acid with at least one primer that includes a phosphorothioate linkage to yield a an amplicon that includes a copy of the target nucleic acid and the phosphorothioate linkage, in which the protein comprises a Cas endonuclease complexed with a guide RNA, wherein the guide RNA comprises a targeting portion that hybridizes to a complementary portion in the copy of the target nucleic acid, and in which digesting the non-target nucleic acid includes exposing the sample to an exonuclease, and in which the exonuclease digests the non-target nucleic acid while the amplicon that include the copy of the target nucleic acid is protected from digestion by the exonuclease by the phosphorothioate linkage and the bound Cas endonuclease.

Optionally, detecting the target nucleic acid includes hybridizing the target nucleic acid to a probe or to a primer for a detection amplification step, or labelling the target nucleic acid with a detectable label.

The methods may include binding the protein to the target nucleic acid, digesting away non-target, dissociating the bound protein, and then amplifying the target nucleic acid by a rolling circle amplification.

### BRIEF DESCRIPTION OF THE DRAWINGS

FIG. 1 diagrams a method a method of detecting a target nucleic acid.

FIG. 2 illustrates obtaining a sample comprising a target nucleic acid.

FIG. 3 shows binding a protein to amplicons containing copies of the mutation.

FIG. 4 shows digesting non-target nucleic acid.

FIG. 5 shows detecting the target nucleic acid and optionally providing a report.

FIG. 6 diagrams a method for detecting a mutation.

FIG. 7 illustrates the operation of allele-specific guide RNA for mutation detection.

FIG. 8 illustrates a negative enrichment.

FIG. 9 shows a kit of the invention.

FIG. 10 illustrates methods of the disclosure.

FIG. 11 diagrams an example in which rolling circle amplification is used in detection.

FIG. 12 shows an example in which rolling circle amplification is used.

### DETAILED DESCRIPTION

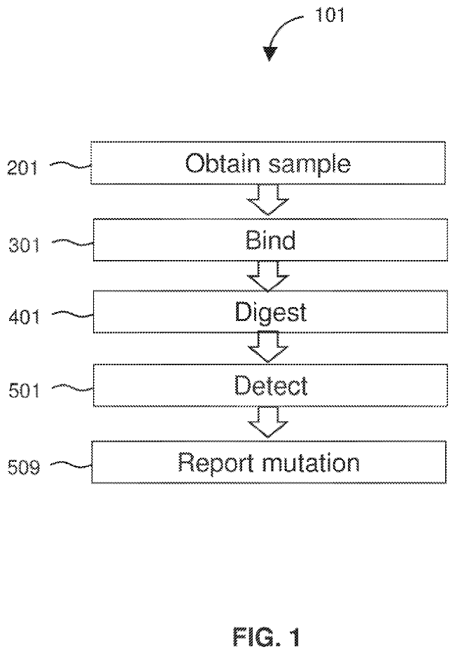

FIG. 1 diagrams a method a method 101 of detecting a target nucleic acid. The method includes obtaining 201 a sample comprising a target nucleic acid, binding 301 a protein to the target nucleic acid in a sequence-specific manner, digesting 401 non-target nucleic acid in the sample, and detecting 501 the target nucleic acid. The method 101 may include providing a report 509 describing the mutation as present in the patient.

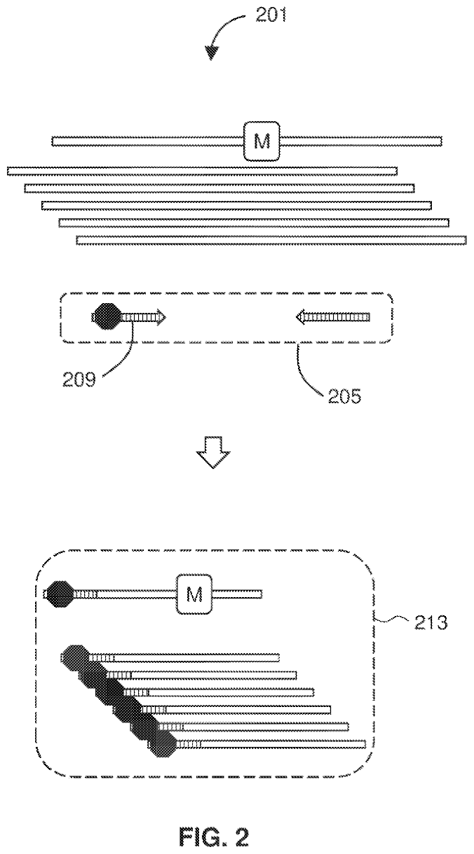

FIG. 2 illustrates obtaining 201 a sample comprising a target nucleic acid. In the depicted embodiment, the target is a mutation (“M”) present only in very small quantities, e.g., even as low as 0.01% frequency of mutant fragments among normal fragments in a sample. A plurality of homologous fragments includes a large number (e.g., >500.000) of wild-type fragments and a small number (e.g., <50) of mutant fragments. Here, methods of the disclosure may have particular applicability in discovering very rare yet clinically important information, such as mutations that are specific to a tumor and even may be used to detect specific mutations among cell-free DNA, such as tumor mutations among circulating tumor DNA. Additionally, in some embodiments, methods of the disclosure may be used to detect rare ribonucleic acids (e.g., in transcripts) using a binding protein that binds to RNA (e.g., a Cas13 enzyme).

The method may include amplifying the target nucleic acid with at least one primer that is resistant to degradation by a nuclease to yield an amplicon that includes a copy of the target nucleic acid and a terminal portion that is resistant to degradation by the nuclease. As shown, a PCR-style amplification is performed using a set of primer pairs 205 in which at least one primer 209 of the pair is resistant to exonuclease activity, e.g., by including a phosphorothioate linkage.

Optionally, another amplification, such as an isothermal amplification, may be used as well as the PCR step (e.g., either before or after) to aid in preserving the small mutant allele fraction. Suitable isothermal amplification methods include recombinase polymerase amplification (RPA) and rolling circle amplification (RCA). For example, in some embodiments, a rolling circle amplification (RCA) is first performed, and the product of the RCA is the substrate for the PCR with on phosphorothioate primer 209. The product of the amplification reaction is a plurality of amplicons 213. Among the amplicons 213, the mutation (“M”) will be present in an allele fraction approximating an allele frequency of the mutation in the original sample. The amplicons 213 are exposed to a binding protein such as a Cas endonuclease.

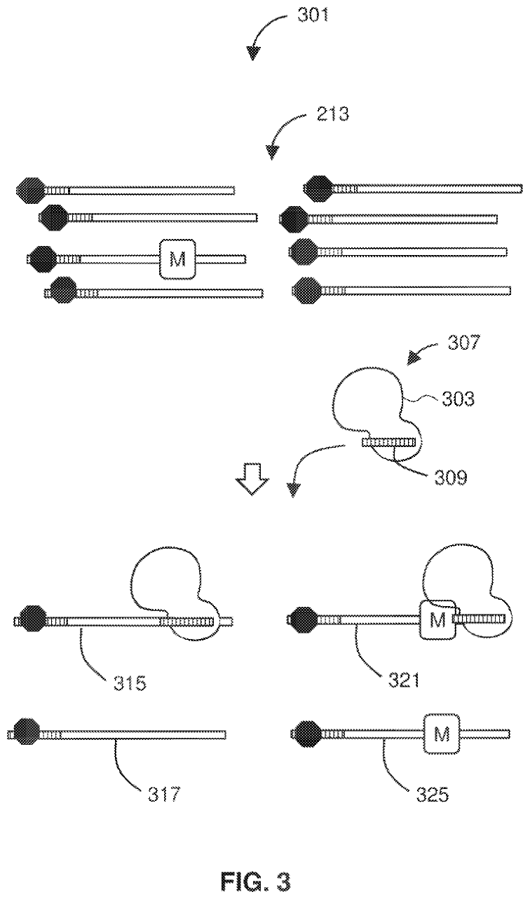

FIG. 3 shows binding 301 a protein 307 to amplicons 213 containing copies of the target nucleic acid in a sequence-specific manner. In a preferred embodiment, the binding proteins 307 are provided by Cas endonuclease/guide RNA complexes. Embodiments of the invention use proteins that are originally encoded by genes that are associated with clustered regularly interspaced short palindromic repeats (CRISPR) in bacterial genomes. Preferred embodiments use a CRISPR-associated (Cas) endonuclease. For such embodiments, the binding protein in a Cas endonuclease complexed with a guide RNA that targets the Cas endonuclease to a specific sequence. Any suitable Cas endonuclease or homolog thereof may be used. A Cas endonuclease (catalytically active or deactivated) may be Cas9 (e.g., spCas9), catalytically inactive Cas (dCas such as dCas9), Cpf1 (aka Cas12a), C2c2, Cas13, Cas13a, Cas13b, e.g., PsnCas13b, LbaCas13a. LwaCas13a, AsCas12a, others, modified variants thereof, and similar proteins or macromolecular complexes. The Cas13 proteins may be preferred where the target includes RNA. A Cas endonuclease/guide RNA complex includes a first Cas endonuclease 303 and a first guide RNA 309.

Binding 301 the binding protein 307 may result in a mixture that includes any or all of bound normal amplicon 315, unbound (“free”) normal amplicon 317, bound mutant amplicon 321, and unbound mutant amplicon 325 copies. This mixture is exposed to, preferably, an exonuclease.

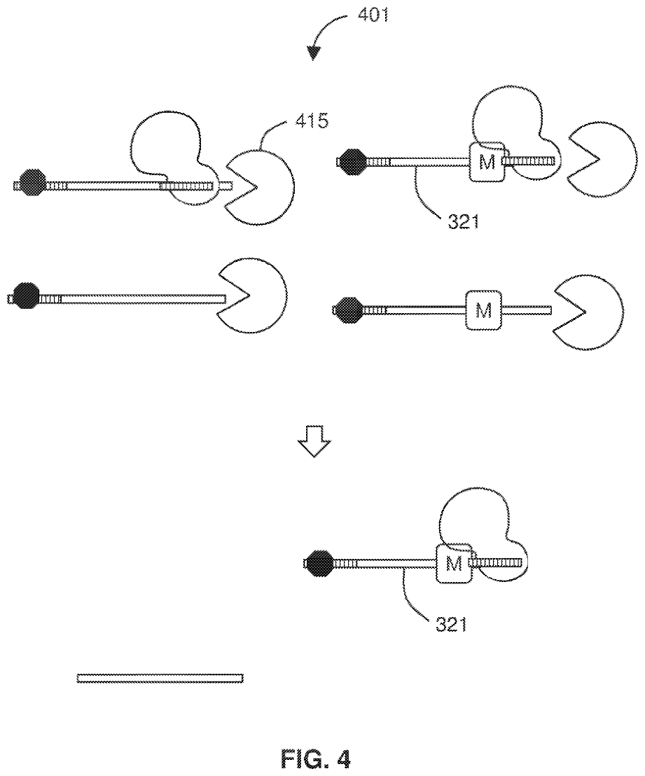

FIG. 4 shows digesting 401 non-target nucleic acid in the sample. An excess of exonuclease 415 is preferably introduced. Unbound normal amplicon 317 and unbound mutant amplicon 325 copies are fully and readily digested by the exonuclease 415. Bound normal amplicon is only protected at one end, so the exonuclease 415 digests those fragments. The bound mutant amplicon 321 is protected at a first end by phosphorothioate linkage and at a second end by the binding protein 301. These fragments are inaccessible to the exonuclease 415 and thus remain as a reaction product 407 after the digesting step 401 and may be detected to the describe the presence of the mutation in the sample.

In certain embodiments, the digesting 401 includes enzymatic digestion by copies of the binding protein 301 that binds 301 to the target nucleic acid. For example, Cas12a may be used as an RNA-guided DNA binding protein and may also operate to indiscriminately cleave single-stranded DNA. A feature of certain Cas endonucleases may be employed by which those proteins use a guide RNA to bind to a target in DNA and, upon binding to target, begin the rapid and complete, indiscriminate cutting of single stranded DNA. Thus methods may use such Cas endonucleases (e.g., LbCas12a or any other Cas12a/Cpf1 enzyme) to bind 301 to the target and also digest 401 non-target nucleic acid in a sample. See Chen, 2018. CRISPR-Cas12a target binding unleashes indiscriminate single-stranded DNase activity, Science 10.1126/science.aar6245, incorporated by reference.

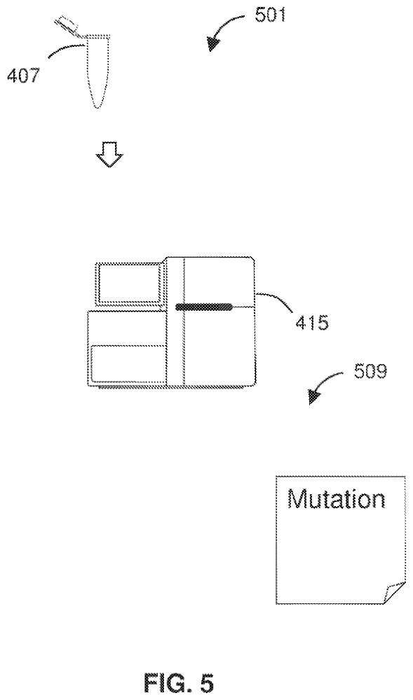

FIG. 5 shows detecting 501 the target nucleic acid and optionally providing a report 509 describing the mutation as present in the patient. The un-digested, mutation-containing fragments may be detected 501 by a suitable assay, such as sequencing, gel electrophoresis, a probe-based assay.

In certain embodiments, the mutation-containing fragments 321 are detected by a detection assay that includes a rolling circle amplification (RCA). RCA is an isothermal nucleic acid amplification technique in which a polymerase continuously adds nucleotides to a primer annealed to a circular template which results in a long concatemer ssDNA that contains tens to hundreds of tandem repeals (complementary to the circular template. Components of a RCA reaction include a DNA polymerase, a suitable buffer that is compatible with the polymerase, a short DNA or RNA primer, a circular DNA template, ad deoxynucleotide triphosphates (dNTPs). Preferred polymerases used in RCA include Phi29, Bst, and Vent exo-DNA polymerase for DNA amplification, and T7 RNA polymerase for RNA amplification. Since Phi29 DNA polymerase has the best processivity and strand displacement ability among all aforementioned polymerases, it has been most frequently used in RCA reactions. Different from polymerase chain reaction (PCR), RCA can be conducted at a constant temperature (room temperature to 37 C) in both free solution and on top of immobilized targets (solid phase amplification). RCA typical includes: circular template ligation, which can be conducted via template mediated enzymatic ligation (e.g., T4 DNA ligase) or template-free ligation using special DNA ligases (i.e., CircLigase); primer-induced single-strand DNA elongation (multiple primers can be employed to hybridize with the same circle; as a result, multiple amplification events can be initiated, producing multiple RCA products and a linear RCA product can be converted into multiple circles using restriction enzyme digestion followed by template mediated enzymatic ligation; and amplification product detection and visualization, which is most commonly conducted through fluorescent detection, with fluorophore-conjugated dNTP, fluorophore-tethered complementary or fluorescently-labeled molecular beacons. In addition to the fluorescent approaches, gel electrophoresis is also widely used for the detection of RCA product. The produced multiple single-stranded linear copies of the target (or ds products thereof) may be desired as they provide a substrate for subsequent detection (e.g., probing) in which the originally “rare” mutation is now present in multiple copies.

The described methods and related kits may be used to detect the presence of mutation in a sample. Due to the nature by which a protein such as a Cas complex binds to a target, methods may be used even where the target is present only in very small quantities, e.g., even as low as 0.01% frequency of mutant fragments among normal fragments in a sample. I.e., where a plurality of homologous fragments includes about 500,000 wild-type fragments and about 50 mutant fragments, methods and kits of the disclosure may usefully detect the presence of the mutant fragments. Thus methods of the disclosure may have particular applicability in discovering and reporting 509 very rare yet clinically important information, such as mutations that are specific to a tumor and even may be used to detect specific mutations among cell-free DNA, such as tumor mutations among circulating tumor DNA.

It is noted that the Cas9/gRNA complexes may be subsequently or previously labeled using standard procedures. The complexes may be fluorescently labeled, e.g., with distinct fluorescent labels such that detecting involves detecting both labels together (e.g., after a dilution into fluid partitions). Preferred embodiments of the detection 501 does not require PCR amplification and therefore significantly reduces cost and sequence bias associated with PCR amplification. Sample analysis can also be performed by a number of approaches such as NGS etc. However, many analytical platforms may require PCR amplification prior to analysis. Therefore, preferred embodiments of analysis of the reaction products include single molecule analysis that avoid the requirement of amplification.

Kits and methods of the invention are useful with methods disclosed in U.S. Provisional Patent Application 62/526,091, filed Jun. 28, 2017, for POLYNUCLEIC ACID MOLECULE ENRICHMENT METHODOLOGIES and U.S. Provisional Patent Application 62/519,051, filed Jun. 13, 2017, for POLYNUCLEIC ACID MOLECULE ENRICHMENT METHODOLOGIES, both incorporated by reference.

FIG. 5 shows the detection 501 of the isolated segment 321 of the nucleic acid. The digestion 401 provides a reaction product 407 that includes principally only the segment 321 of nucleic acid that includes a copy of the mutation of interest, as well as any spent reagents, Cas endonuclease complexes, exonuclease, nucleotide monophosphates, or pyrophosphate as may be present. The reaction product may be provided as an aliquot (e.g., in a micro centrifuge tube such as that sold under the trademark EPPENDORF by Eppendorf North America (Hauppauge, NY) or glass cuvette). The reaction product 407 may be disposed on a substrate. For example, the reaction product may be pipetted onto a glass slide and subsequently combed or dried to extend the fragment 321 across the glass slide. The reaction product may optionally be amplified. Optionally, adaptors are ligated to ends of the reaction product, which adaptors may contain primer sites or sequencing adaptors. The presence of the segment 321 in the reaction product 407 may then be detected using an instrument 415.

The fragment 321 may be detected, sequenced, or counted. Where a plurality of fragment 321 are present or expected, the fragment may be quantified, e.g., by qPCR.

In certain embodiments, the instrument 415 is a spectrophotometer, and the detection 501 includes measuring the adsorption of light by the reaction product 407 to detect the presence of the segment 321. The method 101 may be performed in fluid partitions, such as in droplets on a microfluidic device, such that each detection step is binary (or “digital”). For example, droplets may pass a light source and photodetector on a microfluidic chip and light may be used to detect the presence of a segment of DNA in each droplet (which segment may or may not be amplified as suited to the particular application circumstance). By the described methods, a sample can be assayed for a rare mutation using a technique that is inexpensive, quick, and reliable. Methods of the disclosure are conducive to high throughput embodiments, and may be performed, for example, in droplets on a microfluidic device, to rapidly assay a large number of aliquots from a sample for one or any number of genomic structural alterations.

The Cas endonuclease/guide RNA complexes can be designed to bind to mutations of clinical significance, such as a mutation specific to a tumor. When a mutation is thus detected, a report may be provided 501 to, for example, describe the mutation in a patient.

FIG. 5 shows a report as may be provided in certain embodiments. The report preferably includes a description of the mutation in the subject (e.g., a patient). The method 101 for detecting rare nucleic acid may be used in conjunction with a method of describing mutations (e.g., as described herein). Either or both detection process may be performed over any number of loci in a patient's genome or preferably in a patient's tumor DNA. As such, the report may include a description of a plurality of structural alterations, mutations, or both in the patient's genome or tumor DNA. As such, the report may give a description of a mutational landscape of a tumor.

Knowledge of a mutational landscape of a tumor may be used to inform treatment decisions, monitor therapy, detect remissions, or combinations thereof. For example, where the report includes a description of a plurality of mutations, the report may also include an estimate of a tumor mutation burden (TMB) for a tumor. It may be found that TMB is predictive of success of immunotherapy in treating a tumor, and thus methods described herein may be used for treating a tumor.

Methods of the invention thus may be used to detect and report clinically actionable information about a patient or a tumor in a patient. For example, the method 101 may be used to provide a report describing the presence of the genomic alteration in a genome of a subject. Additionally, protecting a segment 321 of DNA and digesting 401 unprotected DNA provides a method for isolation or enrichment of DNA fragments, i.e., the protected segment. It may be found that the described enrichment technique is well-suited to the isolation/enrichment of arbitrarily long DNA fragments, e.g., thousands to tens of thousands of bases in length.

Long DNA fragment targeted enrichment, or negative enrichment, creates the opportunity of applying long read platforms in clinical diagnostics. Negative enrichment may be used to enrich “representative” genomic regions that can allow an investigator to identify “off rate” when performing CRISPR Cas9 experimentation, as well as enrich for genome regions that would be used to determine TMB for immuno-oncology associated therapeutic treatments. In such applications, the negative enrichment technology is utilized to enrich large regions (>50 kb) within the genome of interest.

In related embodiments, the invention provides methods 101 for detecting structural alterations and/or methods for detecting mutations in DNA.

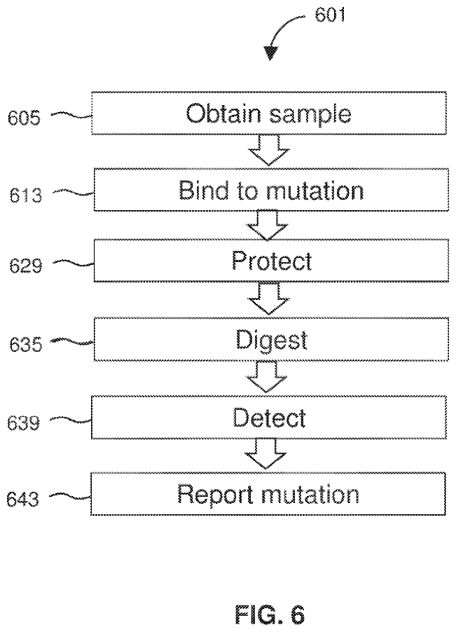

FIG. 6 diagrams a method 601 for detecting a mutation. The method 601 includes obtaining 605 a sample that includes DNA from a subject. The sample is exposed to a first Cas endonuclease/guide RNA complex that binds 613 to a mutation in a sequence-specific fashion. The method 601 includes protecting 629 a segment of nucleic acid in a sample by introducing the first Cas endonuclease/guide RNA complex (that binds to a mutation in the nucleic acid) and a second Cas endonuclease/guide RNA complex that also binds to the nucleic acid. Unprotected nucleic acid is digested 635. For example, one or more exonucleases may be introduced that promiscuously digest unbound, unprotected nucleic acid. While the exonucleases act, the segment containing the mutation of interest is protected by the bound complexes and survives the digestion step 635 intact. The method 601 includes detecting 639 the segment, there confirming the presence of the mutation. A report may be provided 643 that describes the mutation as being present in the subject.

The method 601 uses the idea of mutation-specific gene editing, or “allele-specific” gene editing, which may be implemented via complexes that include at Cas endonuclease and an allele-specific guide RNA.

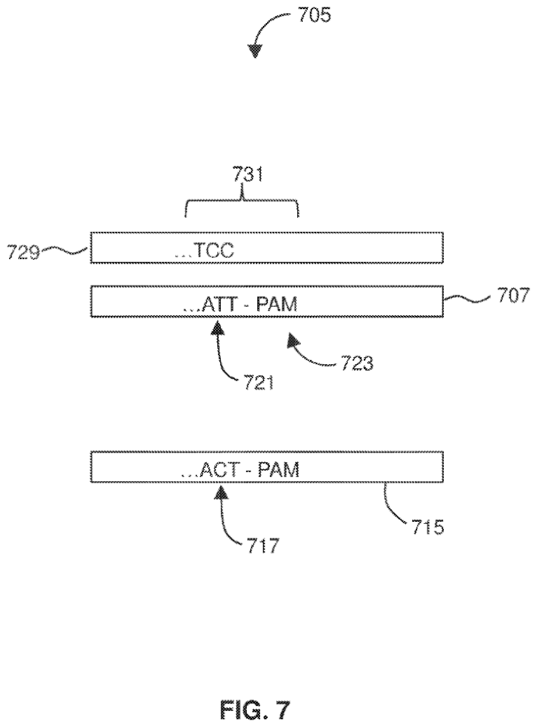

FIG. 7 illustrates the operation of allele-specific guide RNA for mutation detection. A sample 705 may contain a mutant fragment 707 of DNA, a wild-type fragment 715 of DNA, or both. A locus of interest is identified where a mutation 721 may be present proximal to, or within, a protospacer adjacent motif (PAM) 723. When the wild-type fragment 715 is present, it may contain a wild-type allele 717 at a homologous location in the fragment 715, also proximal to, or within, a PAM. A guide RNA 729 is introduced to the sample that has a targeting portion 731 complementary to the portion of the mutant fragment 707 that includes the mutation 721. When a Cas endonuclease is introduced, it will form a complex with the guide RNA 729 and bind to the mutant fragment 707 but not to the wild-type fragment 715. The first Cas endonuclease/guide RNA complex includes a guide RNAs with targeting region that binds to the mutation but that does not bind to other variants at a loci of the mutation.

The described methodology may be used to target a mutation 721 that is proximal to a PAM 723, or it may be used to target and detect a mutation in a PAM, e.g., a loss-of-PAM or gain-of-PAM mutation. The PAM is typically specific to, or defined by, the Cas endonuclease being used. For example, for Streptococcus pyogenes Cas9, the PAM include NGG, and the targeted portion includes the 20 bases immediately 5′ to the PAM. As such, the targetable portion of the DNA includes any twenty-three consecutive bases that terminate in GG or that are mutated to terminate in GG. Such a pattern may be found to be distributed over a genome at such frequency that the potentially detectable mutations are abundant enough as to be representative of mutations over the genome at large. In such cases, allele-specific negative enrichment may be used to detect mutation, in targetable portions of a genome. Moreover, the method 601 may be used to determine a number of mutations over the representative, targetable portion of the genome. Since the targetable portion of the genome is representative of the genome overall, the number of mutations may be used to infer a mutational burden for the genome overall. Where the sample includes tumor DNA and the mutations are detected in tumor DNA, the method 601 may be used to give a tumor mutation burden.

The method 601 includes the described negative enrichment, in which a segment of nucleic acid in a sample is protected 629 by a first Cas endonuclease/guide RNA complex (that binds to a mutation in the nucleic acid) and a second Cas endonuclease/guide RNA complex that also binds to the nucleic acid.

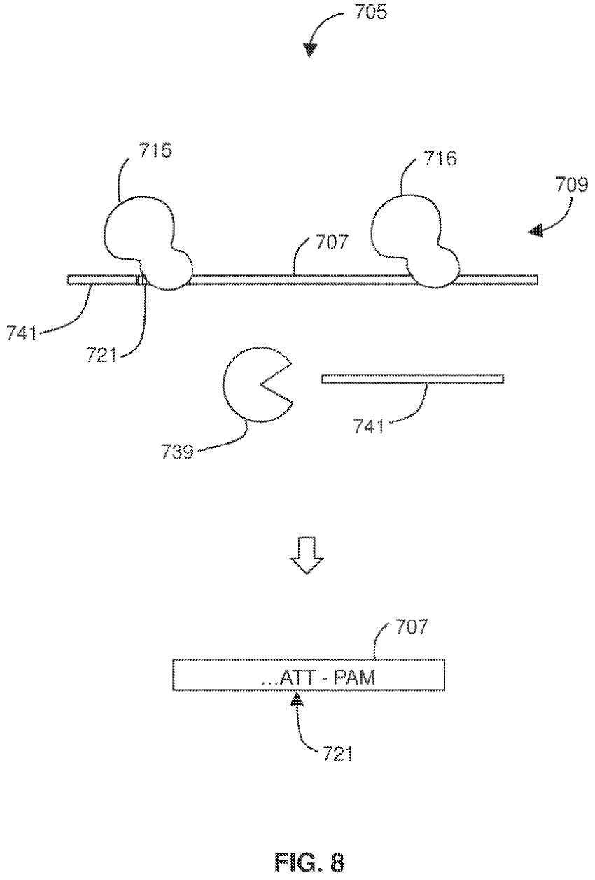

FIG. 8 illustrates operation of the negative enrichment. The sample 705 includes DNA 709 from a subject. The sample 705 is exposed to a first Cas endonuclease/guide RNA complex 715 that binds to a mutant fragment 707 mutation in a sequence-specific fashion. Specifically, the complex 715 binds to the mutation 721 in a sequence-specific manner. A segment of the nucleic acid 709, i.e., the mutant fragment 707, is protected by introducing the first Cas endonuclease/guide RNA complex 715 (that binds to a mutation in the nucleic acid) and a second Cas endonuclease/guide RNA complex 716 that also binds to the nucleic acid. Unprotected nucleic acid 741 is digested. For example, one or more exonucleases 739 may be introduced that promiscuously digest unbound, unprotected nucleic acid 741. While the exonucleases 739 act, the segment containing the mutation of interest, the mutant fragment 707, is protected by the bound complexes 715, 716 and survives the digestion step intact.

The described steps including the digestion by the exonuclease 739 leaves a reaction product that includes principally only the mutant segment 707 of nucleic acid, as well as any spent reagents, Cas endonuclease complexes, exonuclease 739, nucleotide monophosphates, and pyrophosphate as may be present. The method 601 includes detecting 639 the segment 707 (which includes the mutation 721). Any suitable technique may be used to detect 639 the segment 707. For example, detection may be performed using DNA staining, spectrophotometry, sequencing, fluorescent probe hybridization, fluorescence resonance energy transfer, optical microscopy, electron microscopy, others, or combinations thereof. Detecting the mutant segment 707 indicates the presence of the mutation in the subject (i.e., a patient), and the a report may be provided describing the mutation in the patient.

A feature of the method 101 and the method 601 is that a specific mutation may be detected by a technique that includes detecting only the presence or absence of a fragment of DNA, and it need not be necessary to sequence DNA from a subject to describe mutations. The method 601, the method 101, or both may be performed in fluid partitions, such as in droplets on a microfluidic device, such that each detection step is binary (or “digital”). For example, droplets may pass a light source and photodetector on a microfluidic chip and light may be used to detect the presence of a segment of DNA in each droplet (which segment may or may not be amplified as suited to the particular application circumstance).

Methods of the disclosure use protection at one or both ends of DNA segments. The gRNA selects for a known mutation on one end. If it doesn't find the mutation, no protection is provided and the molecule gets digested. The remaining molecules are either counted or sequenced. The method 601 is well suited for the analysis of small portions of DNA, degraded samples, samples in which the target of interest is extremely rare, and particularly for the analysis of maternal serum (e.g., for fetal DNA) or a liquid biopsy (e.g., for ctDNA).

The method 601 and the method 101 include a negative enrichment step that leaves the target loci of interest intact and isolated as a segment of DNA. The methods are useful for the isolation of intact DNA fragments of any arbitrary length and may preferably be used in some embodiments to isolate (or enrich for) arbitrarily long fragments of DNA, e.g., tens, hundreds, thousands, or tens of thousands of bases in length or longer. Long, isolated, intact fragments of DNA may be analyzed by any suitable method such as simple detection (e.g., via staining with ethidium bromide) or by single-molecule sequencing. Embodiments of the invention provide kits that may be used in performing methods described herein.

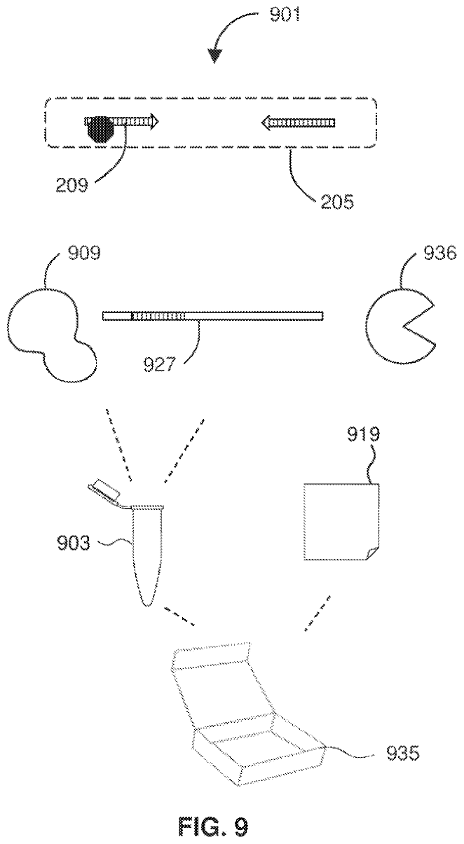

FIG. 9 shows a kit 901 of the invention. The kit 901 may include reagents 903 for performing the steps described herein. The reagents 903 may include one or more set(s) of primer pairs 205 in which at least one primer 209 of the pair is resistant to exonuclease activity, e.g., by including a phosphorothioate linkage. The reagents 903 in the kit 901 may include one or more of a Cas endonuclease 909, a guide RNA 927, and exonuclease 936. The kit 901 may also include instructions 919 or other materials such as pre-formatted report shells that receive information from the methods to provide a report (e.g., by uploading from a computer in a clinical services lab to a server to be accessed by a geneticist in a clinic to use in patient counseling). The reagents 903, instructions 919, and any other useful materials may be packaged in a suitable container 935. Kits of the invention may be made to order. For example, an investigator may use, e.g., an online tool to design guide RNA and reagents for the performance of methods 101, 601. The guide RNAs 927 may be synthesized using a suitable synthesis instrument. The synthesis instrument may be used to synthesize oligonucleotides such as gRNAs or single-guide RNAs (sgRNAs). Any suitable instrument or chemistry may be used to synthesize a gRNA. In some embodiments, the synthesis instrument is the MerMade 4 DNA/RNA synthesizer from Bioautomation (Irving, TX). Such an instrument can synthesize up to 12 different oligonucleotides simultaneously using either 50, 200, or 1,000 nanomole prepacked columns. The synthesis instrument can prepare a large number of guide RNAs 927 per run. These molecules (e.g., oligos) can be made using individual prepacked columns (e.g., arrayed in groups of 96) or well-plates. The resultant reagents 903 (e.g., set(s) of primer pairs 205, guide RNAs 917, endonuclease(s) 909, exonucleases 936) can be packaged in a container 935 for shipping as the kit 901.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

### EXAMPLES

### Example 1

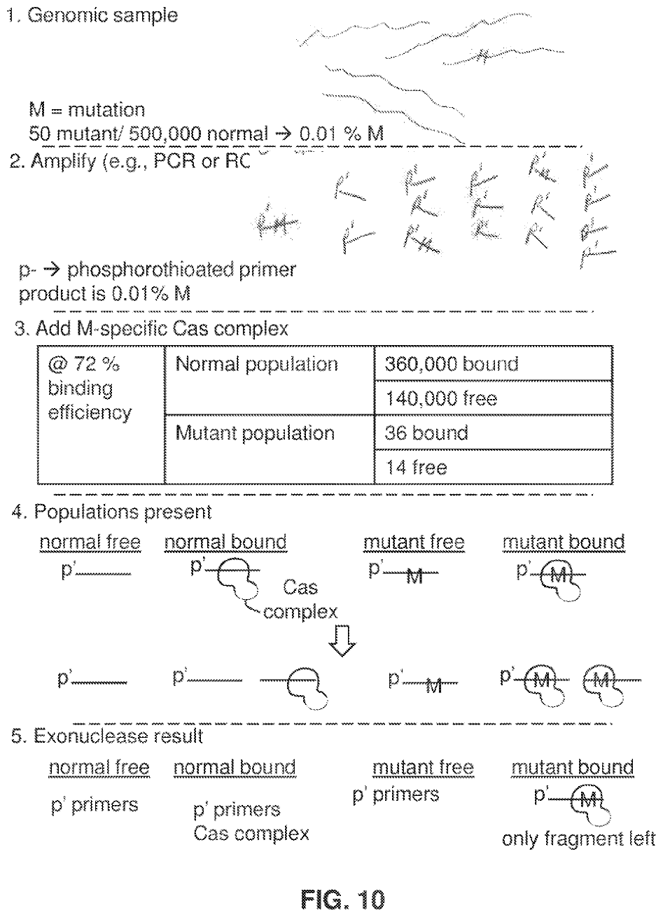

FIG. 10 illustrates methods of the disclosure.

First, a genomic sample is obtained in which about 50 mutant fragments may be present among about 5000,000 homologous wild-type fragments, indicating a mutant frequency of 0.01%.

Second, nucleic acid from the sample may be amplified. It may be preferable to perform a step of a rolling circle amplification. In most preferred embodiments, a PCR-style amplification is performed using a set of primer pairs in which at least one primer of the pair is resistant to exonuclease activity, e.g., by including a phosphorothioate linkage.

The target region is amplified using the primers to yield a plurality of amplicons in which one “sense” of the strands of the amplicons include phosphorothioate linkages near one end of the strand. Among the amplicons, the mutation of interest will be present in an allele fraction approximating an allele frequency of the mutation in the original sample. Thus, as illustrated, the amplicons will have the phosphorothioate linkages at one end and about 0.01% of those amplicons will include a copy of the mutation.

Third, an RNA-guided binding protein such as a Cas complex (e.g., a Cas endonuclease or a catalytically inactivated Cas endonuclease complexed with a guide RNA) is introduced with a guide RNA that binds to the mutation of interest. The Cas complex binds to amplicons that include the mutation. Given the amplification step, for every 500,050 input fragments, the Cas complex will hybridize to mutant and normal amplicons according to a certain characteristic binding efficiency. For example, if the Cas complex binds to target with a 72% binding efficiency, it may be found that for every 500,050 input fragments, introducing the Cas complex results in 360,000 bound normal amplicon, 140,000 unbound (“free”) normal amplicon, 36 bound mutant amplicon, and 14 unbound mutant amplicon copies.

Fourth, the Cas complex is allowed to bind (and cut, when catalytically active Cas is used), which results in a characteristic population of fragments being present after Cas binding. The population of fragments will include unbound normal amplicon Note that Cas may bind in two orientations, where it is catalytically active as an endonuclease, even when it binds normal fragments, it will cleave those fragments and may (in some instances) tend to stay associated with the fragment that does not include the phosphorothioate linkage. Thus the populations present will include fragments with a Cas bound, but no modified backbone bonds.

Some amount of unbound mutant amplicon may be present. And the population of fragments will include some mutant fragments with Cas bound (in either orientation) and, if Cas cleaves, there may be cut fragments with Cas still bound, present both with and without phosphorothioate bonds.

Fifth, the population is treated to ablate non-target nucleic acid (i.e., to ablate fragments of nucleic acid that do not include the mutation. An exonuclease is introduced, preferably one that promiscuously cuts accessible nucleic acid. After a digestion with the exonuclease, fragments of the original primers will remain (due to the phosphorothioate linkages). However, those amplicons that include the mutation and are hound by the Cas complex will not be digested by the exonuclease. For those amplicons, one end of the fragment will be protected from the exonuclease by the phosphorothioate linkages, while the other end will be bound by the Cas complex.

After digestion with the exonuclease, the only fragments that remain will be amplicons that contain the mutation of interest. Those amplicons can then be detected by a suitable assay, such as sequencing, gel electrophoresis, or a probe-based assay. In preferred embodiments, the sample comprises a liquid biopsy sample. The target nucleic acid may include a mutation specific to a tumor. The tumor mutation may be present at no more than about 0.01% among matched normal, non-tumor nucleic acid.

### Example 2

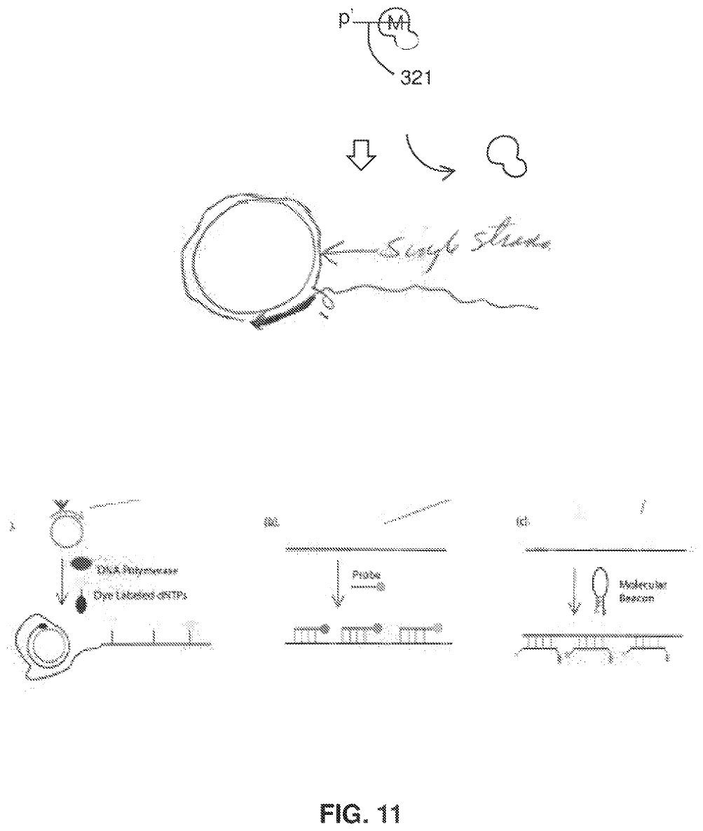

FIG. 11 diagrams an example in which rolling circle amplification is used in detection.

The example begins with fragments 321, which are produced by the negative enrichment steps of method 101 and by example 1 and which may be used for the analysis of cancer mutations in plasma samples.

In this example 2, the process includes those same steps as described above for method 101 and for example 1. Those steps yield (besides negligible spent reagents, dNTPs, buffer salts, etc.) only mutation specific and protected DNA fragments 321 from the patient sample.

As shown in FIG. 11, the p′ indicates the phosphorthioate base(s) incorporated into the fragments 321 from the original PCR primers, protecting a first end of the fragments 321. The “M” is the rare mutation, here shown bound by mutation-specific Cas complex, which is protecting a second end of the fragments 321.

The Cas endonuclease complexes are dissociated from the fragments 321, and the remaining fragments are incorporated into an RCA reaction. For example, a ligation template may be used which includes portions complementary to the phosphorothioate primer and matching the targeting portion of the guide RNA.

The ends of the fragments 321 will hybridize to the ligation template adjacent each other and in the same 5′ to 3′ orientation as each other. A ligase circularizes the fragments 321 and the ligation template is melted away. An RCA amplification primer is introduced with dNTPs and a DNA polymerase, which extends the primer and gives a linear reaction product with many copies of the rare mutation (which may actually be in the same sense as the original mutation, or may be a reverse complement to the original mutation). In preferred embodiments, the polymerase is Phi29 which extends around the circularized template and displaces the primer and the nascent linear product, making the large number of copies.

The resultant synthesized RCA product may be detected by any suitable method including, for example, having labeled bases directly incorporated into the RCA product, or by having labeled probes in the mix that hybridize to the ssDNA (e.g., steps b and c in FIG. 11).

### Example 3

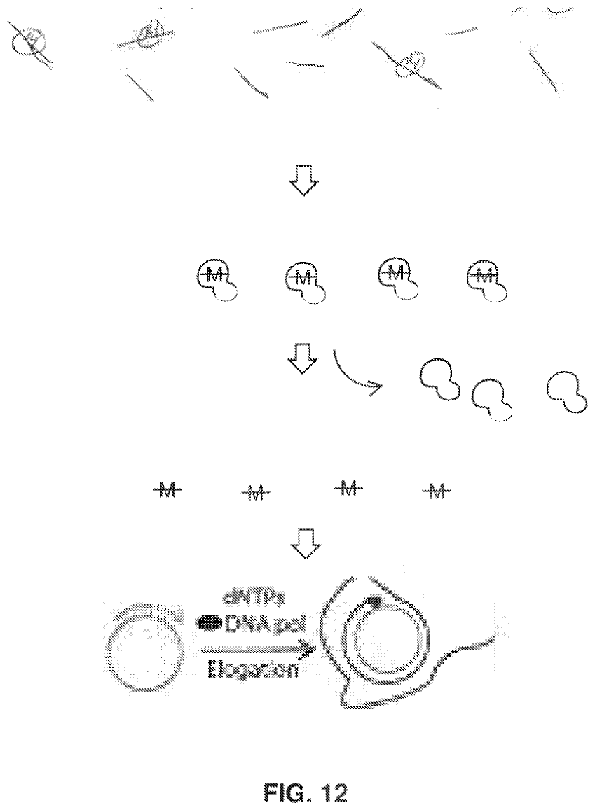

FIG. 12 shows an example in which rolling circle amplification is used. In the depicted example 3, nucleic acid is obtained from a sample such as plasma and the amplification step of method 101 (diagramed in FIG. 2) is skipped or omitted. The nucleic may be purified, e.g., from plasma and mutation-specific Cas complexes am directly added. This example may be beneficial when the target nucleic acid includes RNA as a Cas13 protein may be added (e.g., complexed with a guide RNA as a ribonucleoprotein (RNP)). See Gootenberg, 2018, Multiplexed and portable nucleic acid detection platform with Cas13, Cas12a, and Csm6, Science 10.1126/science.aaq0179, incorporated by reference. Where the target is DNA, any other suitable Cas endonuclease may be used. Those proteins may be used in combination to target RNA and DNA. In a preferred embodiment, genomic DNA is targeted in a plasma sample, e.g., from a liquid biopsy, to identify rare tumor-specific mutations.

The top panel of FIG. 12 represents the genomic DNA purified from plasma. Mutation-specific Cas complexes are added, which bind to the rare mutations. An exonuclease is introduced, which digests all unprotected DNA. The result is shown in the second panel of FIG. 12. What remains is a plurality of mutation-specific Cas complexes that are each bound to a short (approx. 20 base) segment of the original genomic DNA, in which that bound segment includes the rare mutation.

The Cas complexes are dissociated and resultant product is shown in the third panel of FIG. 12. That resultant product includes a plurality of short (approx. 20 base) segments of the original genomic DNA that include the rare mutation.

Those final approximately 20mer short mutant fragments are added to a rolling circle amplification reaction mix. Optionally, adapters may be ligated to the short mutant fragments to give the RCA ligation template greater purchase for the circularization reaction. The fragments are circularized, preferably with a single-stranded template or adapter, and the single stranded template (and/or the adapter) may be engineered to include a mutation-specific priming site. The circularized product that includes the original rare mutation (which may optionally be present in the original, forward sense). That circularized product is subject to RCA and any detection such as those described above.

What is claimed is:

1. A method of detecting a target nucleic acid, the method comprising:
obtaining a sample comprising a target nucleic acid;
binding a protein to the target nucleic acid in a sequence-specific manner;
digesting non-target nucleic acid in the sample; and
detecting the target nucleic acid.

2. The method of claim 1, further comprising amplifying the target nucleic acid with at least one primer that is resistant to degradation by a nuclease to yield an amplicon that includes a copy of the target nucleic acid and a terminal portion that is resistant to degradation by the nuclease.

3. The method of claim 2, wherein digesting the non-target nucleic acid includes exposing amplicons to the nuclease.

4. The method of claim 3, wherein the nuclease digests the non-target nucleic acid while the amplicon that includes the copy of the target nucleic acid is protected by the terminal portions and the bound protein.

5. The method of claim 2, wherein the at least one primer that is resistant to degradation by a nuclease comprises an oligonucleotide with one or more phosphorothioate linkage.

6. The method of claim 1, wherein the protein comprises an RNA-guided protein complexed with a guide RNA, the guide RNA comprising a targeting portion that hybridizes to a complementary portion in the copy of the target nucleic acid.

7. The method of claim 6, wherein the RNA-guided protein comprises a Cas endonuclease or a catalytically deficient homolog thereof.

8. The method of claim 1, wherein the target nucleic acid includes a mutation, and the sample further includes homologous non-mutated nucleic acid, and the digesting step includes digesting the homologous non-mutated nucleic acid, amplified copies thereof, or both.

9. The method of claim 8, wherein the sample is from a patient, and method includes providing a report describing the mutation as present in the patient.

10. The method of claim 9, further comprising identifying a treatment based on the presence of the mutation in the patient and including the identified treatment option in the report.

11. The method of claim 1, wherein the digesting is performed with an exonuclease.

12. The method of claim 1, wherein the protein comprises a Cas endonuclease complexed with a guide RNA, wherein the guide RNA comprises a targeting portion that hybridizes to a complementary portion in the target nucleic acid.

13. The method of claim 1, wherein the protein comprises a transcription-activator like effector (TALE).

14. The method of claim 1, further comprising
amplifying the target nucleic acid with at least one primer that includes a phosphorothioate linkage to yield a an amplicon that includes a copy of the target nucleic acid and the phosphorothioate linkage,
wherein the protein comprises a Cas endonuclease complexed with a guide RNA, wherein the guide RNA comprises a targeting portion that hybridizes to a complementary portion in the copy of the target nucleic acid,
wherein digesting the non-target nucleic acid includes exposing the sample to an exonuclease,
wherein the exonuclease digests the non-target nucleic acid while the amplicon that include the copy of the target nucleic acid is protected from digestion by the exonuclease by the phosphorothioate linkage and the bound Cas endonuclease.

15. The method of claim 1, wherein detecting the target nucleic acid includes hybridizing the target nucleic acid to a probe or to a primer for a detection amplification step, or labelling the target nucleic acid with a detectable label.

16. The method of claim 1, further comprising binding the protein to the target nucleic acid, digesting away non-target, dissociating the bound protein, and amplifying the target nucleic acid by a rolling circle amplification.

17. The method of claim 1, wherein the sample comprises a liquid biopsy sample.

18. The method of claim 17, wherein the target nucleic acid includes a mutation specific to a tumor.

19. The method of claim 18, when the tumor mutation is present at no more than about 0.01% among matched normal, non-tumor nucleic acid.
